(19) United States
(12) Patent Application Publication
(10) Pub. No.: US 2013/0184580 A1
(43) Pub. Date: Jul. 18, 2013

(54) COLOR FLOW IMAGE AND SPECTROGRAM ULTRASOUND SIGNAL SHARING

(71) Applicant: Brian Anthony Lause, Milwaukee, WI (US)

(72) Inventor: Brian Anthony Lause, Milwaukee, WI (US)

(73) Assignee: GENERAL ELECTRIC COMPANY, Schenectady, NY (US)

(21) Appl. No.: 13/350,503

(22) Filed: Jan. 13, 2012

(51) Int. Cl. A61B 8/00

(52) U.S. Cl. 600/440

(57) ABSTRACT

A color flow image and a spectrogram are generated using the same acquired ultrasound signal.

### BACKGROUND

Ultrasound or ultrasonography is a medical imaging technique that utilizes high-frequency (ultrasound) waves and their reflections. A computer interprets the reflections and presents information for viewing. Examples of modes by which such information may be presented include a brightness mode (B-mode), a color flow or color Doppler mode and a spectral or pulsed wave Doppler mode. Some ultrasound systems offer a duplex mode in which the B-mode and the color flow mode are concurrently presented. Some ultrasound systems offer a triplex mode in which each of the B-mode, the color flow mode and the spectral mode are concurrently presented. Each of the duplex mode and the triplex modes utilize independent ultrasound signals for the concurrently displayed modes. Acquiring the independent ultrasound signals consumes time and processing power.

### BRIEF DESCRIPTION OF THE DRAWINGS

FIG. 1 is a schematic illustration of an example ultrasound system.

FIG. 2 is a flow diagram of an example method that may be carried out by the ultrasound system of FIG. 1.

FIG. 3 is a diagram illustrating a portion of an example spectrogram that may be formed according to the method of FIG. 2.

FIG. 4 is a flow diagram of another example method that may be carried out by the ultrasound system of FIG. 1.

FIG. 5 is a diagram illustrating a portion of an example spectrogram that may be formed according to the method of FIG. 4.

FIG. 6 is a diagram illustrating processing of color flow image ultrasound signals to generate the spectrogram of FIG. 5.

FIG. 7 is a diagram illustrating one example method for processing a packet or set of ultrasound signals in the generation of the spectrogram of FIG. 5.

FIG. 8 is a flow diagram of an example method for generating multiple spectrograms for different locations using a stored color flow image.

### DETAILED DESCRIPTION OF THE EXAMPLE EMBODIMENTS

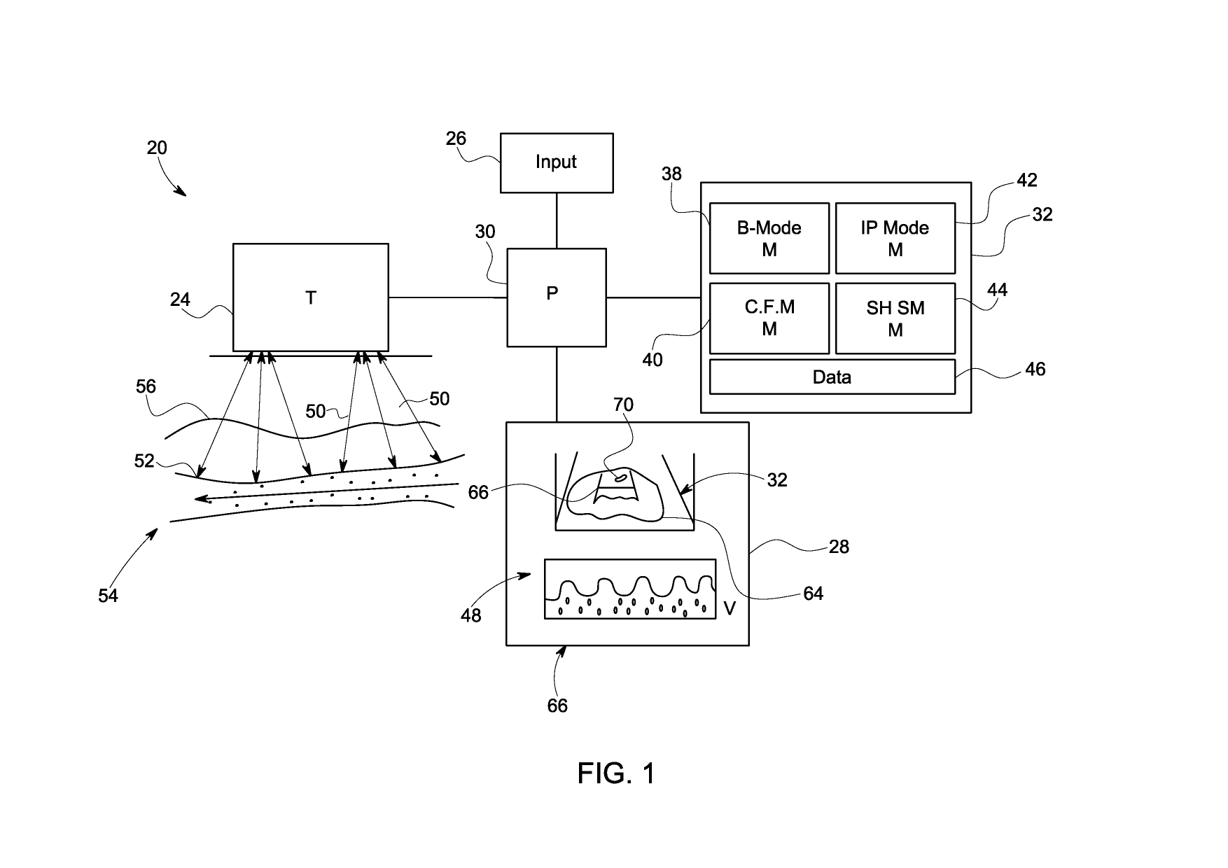

FIG. 1 schematically illustrates an example ultrasound system 20. As will be described hereafter, ultrasound system 20 utilizes the same ultrasound signals and the same ultrasound acquisition sequence for generating both a color flow image and a spectrogram (also known as spectral Doppler or spectral sonogram). As a result, system 20 may concurrently provide both display modes with reduced acquisition and processing times and with enhanced frame rates for the color flow image. In addition, system 20 facilitates (1) the generation of a spectrogram from many spatial locations of the color flow image and (2) the generation of spectrograms from previously generated and stored color flow images.

Ultrasound system 20 comprises transducer 24, input 26, display 28, processor 30 and memory 32. Transducer 24 comprises quartz crystals, piezoelectric crystals, that change shape in response to the application electrical current so as to produce vibrations or sound waves. Likewise, the impact of sound or pressure waves upon such crystals produce electrical currents. As a result, such crystals are used to send and receive sound waves. The received sound waves constitute ultrasound signals which are transmitted to processor 34 for analysis. Such transmission may occur in either a wired or wireless fashion.

Transducer 24 may be housed as part of a handheld ultrasound probe (not shown). The handheld probe may additionally include a sound absorbing substance to eliminate back reflections from the probe itself and an acoustic lens to focus emitted sound waves. Examples of transducer 24 include, but are not limited to, a linear transducer, a sector transducer, a curved transducer and the like.

Input 26 comprises one or more input devices by which a person may enter commands, selections or data into system 20. Examples of input 26 comprise, but are not limited to, a keyboard, mouse, a touchpad, touchscreen, microphone with speech recognition programming, keypad, pushbuttons, slider bars and the like. As will be described hereafter, input 26 enables the input of mode preferences for transducer 26 and for the display of information on output 26.

Display 28 comprises a monitor or display screen by which information based upon the received ultrasound signals is visibly presented. In one implementation, display 28 may be incorporated as part of an overall host, house with processor 30, memory 32 and possibly input 26. In another implementation, display 28 may comprise a separate or independent display screen connected to a host which provides processor 30 and memory 32. In some implementations, display screen 28 may be incorporated as part of the handheld probe itself. In some implementations, display 28 may comprise a touchscreen so as to also serve as input 26.

Processor 30 comprises one or more processing units configured to (1) generate control signals directing the operation of transducer 24, (2) process signals received from transducer 24 and (3) generate control signals directing display 28 to present information based upon the processed signals. In some implementations, such function may perform by multiple independent processors which cooperate with one another. For purposes of this application, the term “processing unit” shall mean a presently developed or future developed processing unit that executes sequences of instructions contained in a non-transient computer-readable program or memory 32. Execution of the sequences of instructions causes the processing unit to perform steps such as generating control signals. The instructions may be loaded in a random access memory (RAM) for execution by the processing unit from a read only memory (ROM), a mass storage device, or some other persistent storage. In other embodiments, hard wired circuitry may be used in place of or in combination with software instructions to implement the functions described. For example, at least portions of processor 30 and memory 32 may be embodied as part of one or more application-specific integrated circuits (ASICs) or programmed logic devices (PLDs). Unless otherwise specifically noted, processor 30 and memory 32 are not limited to any specific combination of hardware circuitry and software, nor to any particular source for the instructions executed by the processing unit. Moreover, processor 30 and memory 32 may be provided using multiple separate sub processes or sub memories that cooperate with one another. For example, a handheld probe may include a processor and memory that carry out some of the functions of system 20 while a monitor or host may include a processor and memory which carry out another portion of the functions of system 20.

Memory 32 comprises a non-transient computer-readable medium containing code provided as software or circuitry for instructing or directing processor 30. Memory 32 comprises brightness mode (B-mode) module 38, color flow mode module 40, spectral mode module 42, shared signal mode module 44 and data region 46. Each of modules 36, 38, 40, 42 and 44 comprises a non-transient computer readable program or code stored in memory 32 and configured to direct processor 30 to acquire and process ultrasound signals so as to display ultrasound imaging information using one or more modes which may be selected by a caretaker using input 26.

B-mode module 38 directs processor 30 to generate control signals causing transducer 24 to transmit and receive ultrasound signals (also known as pulses or waves) and to process the received signals or echo signals to display an image of an anatomy or object on display 28. In one implementation, B-mode module 38 provides a two-dimensional image. In yet other implementations, module 38 may alternatively utilize transducer 24 to present a three-dimensional or four dimensional image of the anatomy or object. In operation, ultrasound signals are scanned across an anatomical area, wherein reflections of such signals are sensed to generate the image.

Color flow mode module 40 directs processor 30 to generate control signals causing transducer 24 to transmit and receive packets or sets of ultrasound signals (sometimes referred to as firings) at each of a matrix of locations in a region of interest. In other words, processor 30 directs transducer 24 to scan across the region of interest, emitting and receiving a set or packet of ultrasound signals at each individual location. The scan across the entire region of interest (in both X and Y directions) provide signals and data which are used to form a single frame of the color flow image being displayed. For purposes of this disclosure, a signal maybe the raw signal itself or may be another signal or data derived from the raw signal. When a frame of the color flow image has been completed, color flow mode module 40 directs processor 30 to generate control signals causing transducer 24 to repeat the previous scan to form successive frames which indicate any change in the color flow image. In one implementation, such scanning is completed at a rate such that display 28 may present a color flow image having a frame rate of at least 5 Hz.

The color flow image generated from the analysis of the received packets or sets of ultrasound signals by processor 30 using Doppler analysis may identify a direction and general qualitative speed of movement of a target of interest, such as blood flow. This direction and qualitative speed is indicated by color and/or brightness on display 28. In one implementation color may be used to indicate direction of flow of brightness may be used to indicate the qualitative or relative speed. The color flow images produced by color flow module 40 provide an overall view of flow in a region of interest, indicating general flow direction, turbulent flows and course speed indications.

Spectral mode module 42 directs processor 30 to generate control signals causing transducer 24 to transmit and receive or acquire ultrasound signals from a single location as selected or identified by a movable icon on display 28 in the form of a cursor, window or gate. Spectral mode module 42 further directs processor 30 to process and analyze the received ultrasound echo signals from the single site or location so as to present a spectrogram on display 28. A spectrogram, also known as a spectral or pulsed wave Doppler or spectral sonogram, is a graph or picture generally indicating a range of blood flow velocities within the gate and a distribution of power over the velocities within the range. By way of comparison, spectral mode module 42 directs transducer 24 to transmit and receive a much larger set of ultrasound signals at the single site or location while color flow mode module 40 directs transducer 24 to transmit and receive a much smaller set of ultrasound signals, but at each of a multitude of locations so as to form a color flow image. In one implementation, spectral mode module 42 directs transducer 24 to transmit and receive over 100 ultrasound signals (nominally 128 or 256 ultrasound signals) at the single location defined by the gate, whereas color flow mode module 40 directs transducer 24 to transmit and receive less than 100 ultrasound signals (less than or equal to 32 pulses or signals in one implementation) (nominally 8, 16 or 32 ultrasound signals) at each location of the matrix of locations which are to be covered or represented by the resulting color flow image.

In some implementations, a caretaker or sonographer may be provided with an option of selecting one or more multi-modes, wherein multiple modes of imaging information are concurrently presented on the display. For example, in one multimode sometimes referred to as a duplex mode, system 20 may be operated in both the B-mode and the color flow mode, wherein the color flow image generated by color flow mode module 40 is superimposed upon the generally larger anatomical image generated by B-mode module 38. In such a case, the acquisition of ultrasound signals by transducer 24 alternates between the acquisition of ultrasound signals under the direction of the B-mode module 38 and color flow mode module 40, with the B-mode image and the color flow image being generated from independent sets of ultrasound signals.

In another multimode sometimes referred to as a triplex mode, system 20 may be operated in each of the B-mode, the color flow mode and the spectral mode. In the triplex mode, the color flow mode image is superimposed upon the B-mode image as described above. In addition, the spectral image or spectrogram is concurrently presented on display 28. In the triplex mode, transducer 24 alternates between the acquisition of ultrasound signals under the direction of the B-mode module 38, color flow mode module 40 and spectral mode module 42 (using time interleaving), with the B-mode image, the color flow image and the spectrogram being generated from independent sets of ultrasound signals acquired independently from one another using transducer 24. During acquisition of the much larger number of ultrasound signals at the single location defined by the gate for the generation of the spectrogram, the frame rate at which the color flow image and the B-mode image may be slowed or frozen.

Shared signal mode module 44 comprises code or programming on a non-transient computer-readable medium such as memory 32 which facilitates the generation of a spectrogram using the same set or sets of ultrasound signals acquired for the generation of the color flow image by color flow mode module 40. In other words, the same set or sets of ultrasound signals are shared by both color flow mode module 40 to generate a color flow image and shared signal mode module 44 to generate a spectrogram. As a result, system 20 may concurrently provide both color flow and the spectral display modes with reduced acquisition and processing times and with enhanced frame rates for the color flow image. In addition, system 20 facilitates (1) the generation of a spectrogram at each of multiple spatial locations of the color flow image and (2) the generation of spectrograms from previously generated and stored color flow images.

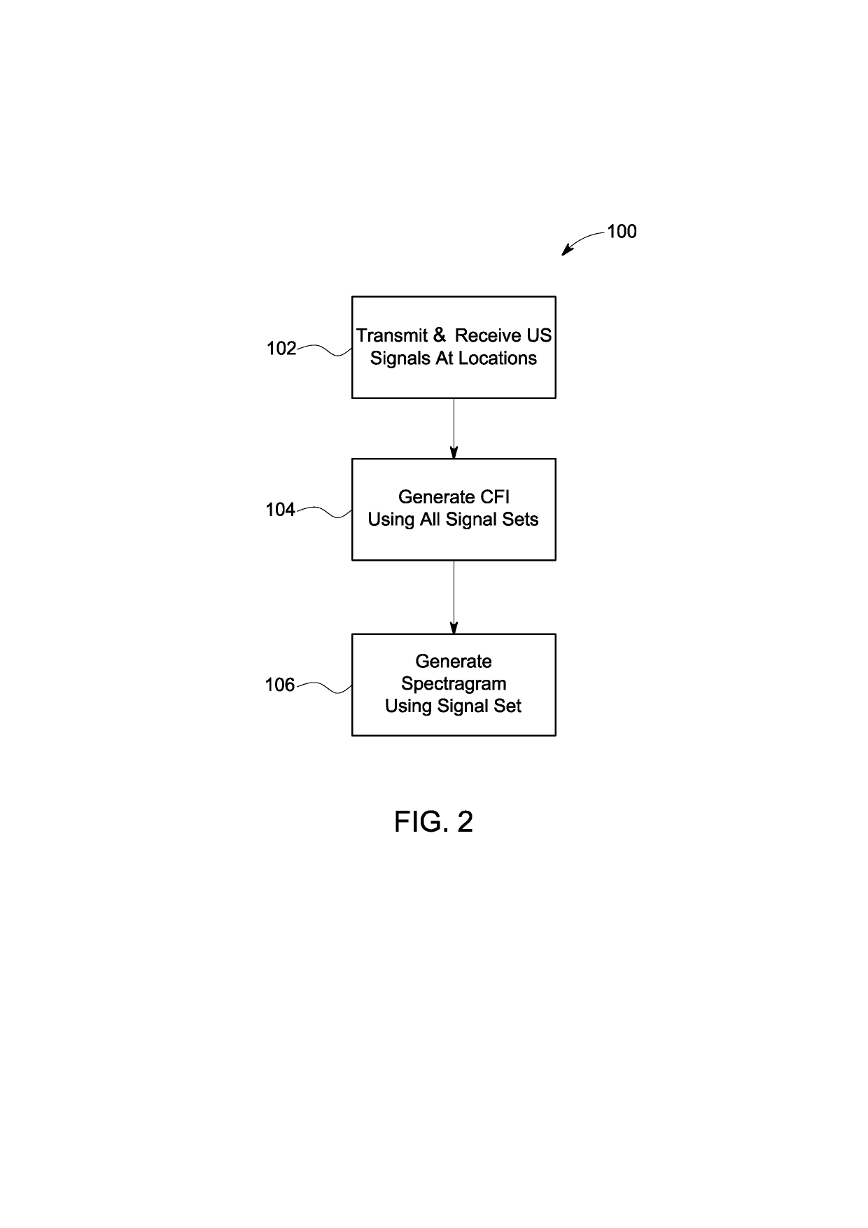

In the example illustrated, shared signal mode module 44 operates in conjunction with both B-mode module 38 and color flow mode module 40 to carry out method 100 shown in FIG. 2 to provide the example presentation 48 of information shown on display 28. As indicated by step 102 in FIG. 2, color flow mode module 40 directs processor 30 to generate control signals causing transducer 24 to operate in a color flow mode as described above with respect to color flow mode module 40, wherein transducer 24 transmits and receives a packet or set of ultrasound signals from each location of a matrix of locations constituting a region of interest to form a color image frame of the region of interest. In particular, as schematically shown by arrows 50 of FIG. 1, transducer 24 directs ultrasound signals or pulses at each of multiple locations 52 in a region of interest 54 which may be a two-dimensional area. In the example illustrated, the B-mode module 38 may also be operating to alternately control transducer 24 (using a time interleaving technique) so as to transmit and receive signals generally across an area greater than the region of interest for the color flow image being acquired by module 40.

As indicated by step 104 in FIG. 2, color flow mode module 40 further directs processor 30 to process and analyze each packet or set echoes, reflections or signals received by transducer 24 at each location of the region of interest to generate a color flow image 60 (shown on display 28 in FIG. 1). In one example, the received echo signals are first modulated and then transformed from a time domain to a frequency domain which corresponds to velocity. To transform such echo signals from the time domain to a frequency domain or to a domain related to frequency, color flow mode module 40 may utilize one of various transforms such as a Kasai analysis, fast Fourier transform, a wavelet transform or a discrete cosine transform. Once these signals have been transformed to a frequency or frequency related domain, frequency or frequency related signals may further be processed to generate color flow image 60.

In the example illustrated, B-mode module 38 also directs processor 30 to process and analyze its ultrasound signals to generate the B-mode image 62 which encompasses an anatomical structure 64. As shown by FIG. 1, in one example implementation, the color flow image 60 is superimposed upon the B-mode image 62 by alternately depicting updated images 60 and 62 on display 28 at a sufficiently high frame rate such that they appear to be superimposed. In other implementations, the operation of B-mode module 38 and the presentation of its B-mode image 62 may be omitted in steps 102 and 104.

As indicated by step 106, shared signal mode module 44 utilizes the same signals used to form color flow image 60 to generate a spectrogram, an example 66 of which is shown in FIG. 1. In particular, module 44 prompts a person to locate gate 70 (or another graphical icon defining a specific site or location for which a spectrogram is to be generated) in the color flow image 60. Module 44 then utilizes the packets or sets of ultrasound signals which were transmitted and received from the particular site or location to generate spectrograph 66 using the algorithms as employed by specular mode module 42.

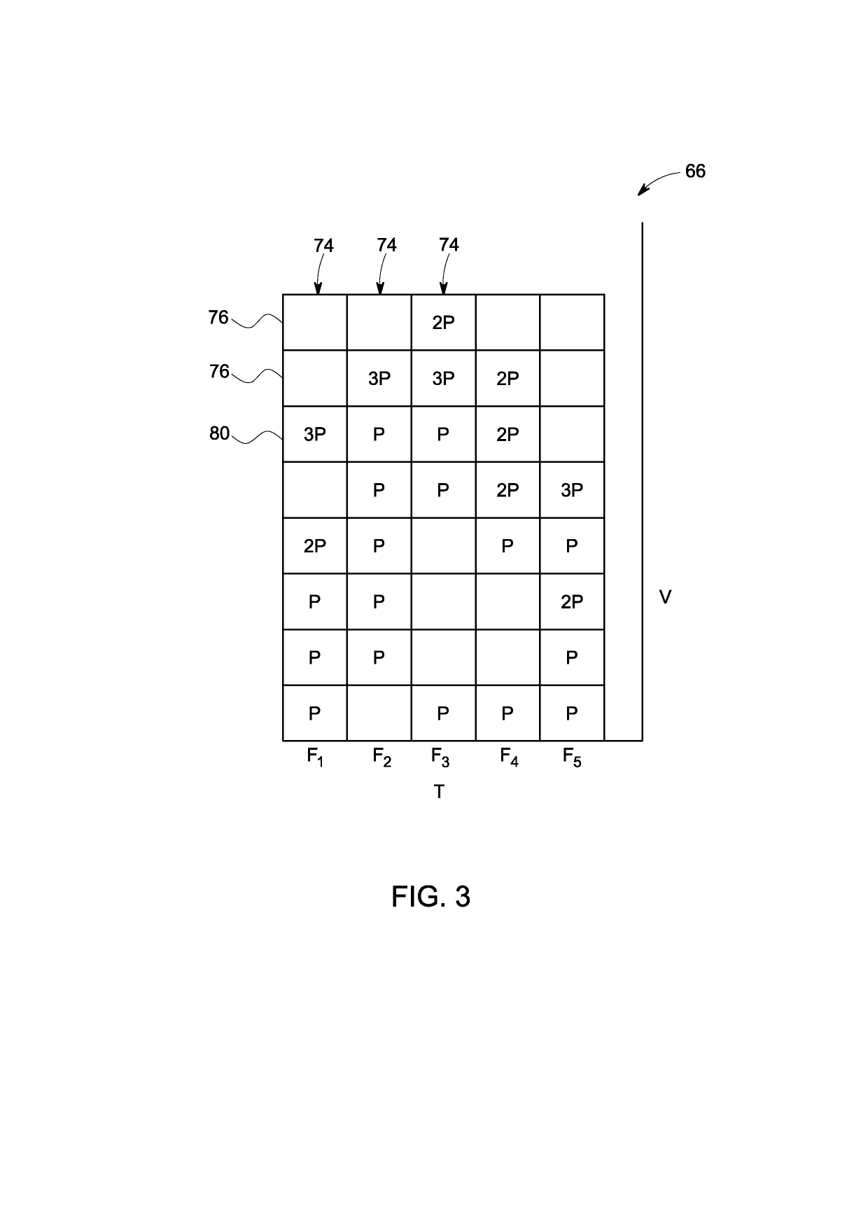

FIG. 3 is a diagram illustrating a portion of spectrogram 66 formed in step 106. As shown by FIG. 3, spectrogram 66 is composed of a series of spectral distribution bars 74. Each bar 74 has a number of segments or bins 76. In one implementation, the number of bins 76 is equal to the number of signals or pulses in each set or packet of signals transmitted and received from each location as part of the color flow mode. In the example illustrated, color flow mode module 40 directs transducer 24 to transmit and receive eight ultrasound signals or pulses at each location for each frame. As a result, each bar 74 has eight segments or bins 76. In other examples where the packet size may be larger or smaller, the number of segments 76 in each bar 74 may also be correspondingly larger or smaller.

Each segment 76 has a brightness indicating a power for the representative frequencies for that segment of reflection exhibited by a particular received echo signal. During each frame, module 44 determines a power for each of the many velocities of a received signal and places the signal in one of bins 76 corresponding to the appropriate velocity. The brightness of each bin 76 corresponds to the power of the pulses or signals of the received signals that correspond to a velocity within the range of the particular bin 76. In the example illustrated, during a frame F₁, the received signal was determined to have velocities within the range of the velocities represented by bin 80 with a power of 3 times some power unit. Bin 80 also represents the highest strength power for any velocity exhibited in the set of signals for the single location defined by gate 70 of FIG. 1. As shown by FIG. 3, module 44 generates a new bar 74 for each frame of the color flow image 60 using the set of ultrasound signals transmitted to and received from a particular location during generation of the frame.

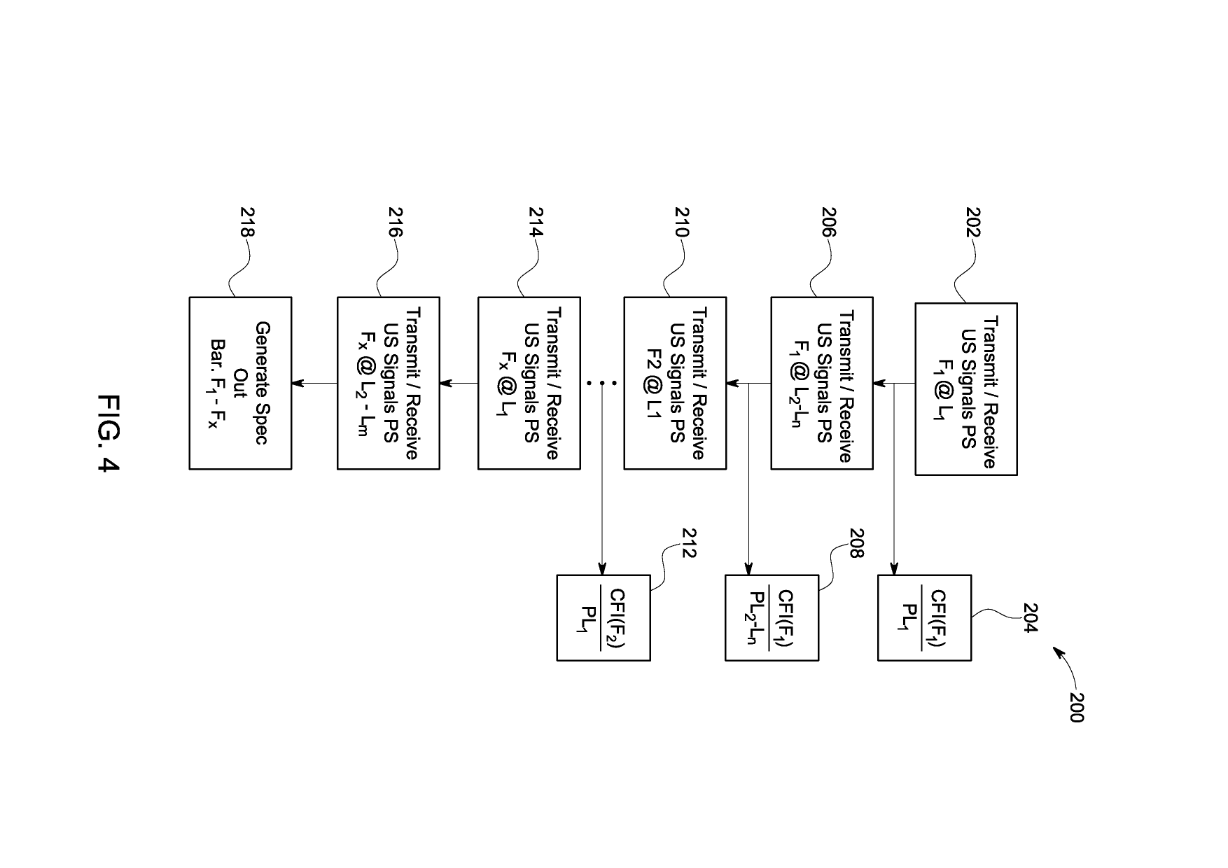

FIG. 4 is a flow diagram illustrating method 200, another example method by which color flow mode module 40 and shared signal mode module 44 may form a color flow image and a spectrogram using the same sets of ultrasound signals. In the example illustrated, the location for which a spectrogram is to be generated (as defined by gate 70 in FIG. 1) is that location L₁. The region of interest or the area to be depicted by a color flow image is a matrix of locations including L₁ to Lₙ. Method 200 is similar to method 100 except that in method 200, each spectral distribution bar of the spectrogram is formed from and based upon a part or whole of a packet or set of signals or pulses from each of a plurality of frames for a pixel or single location of the color flow image. As a result, frequency resolution of the generated spectrogram 266 for the particular location may be enhanced and is not fixed to the packet size used in color flow.

As indicated by step 202, color flow mode module 40 directs or instructs processor 30 to transmit and receive a first set of ultrasound signals of a packet or set size (PS) at a first location L₁ during a first frame F₁ of the color flow image. There may be additional unrelated transmit and receive events interleaved with the acquisition for position L1. As indicated by step 204, color flow mode module 40 (shown in FIG. 1) directs processor 30 to analyze such signals to produce an individual pixel PL₁ at location L₁. This pixel is part of a matrix of pixels that forms the first frame of the color flow image.

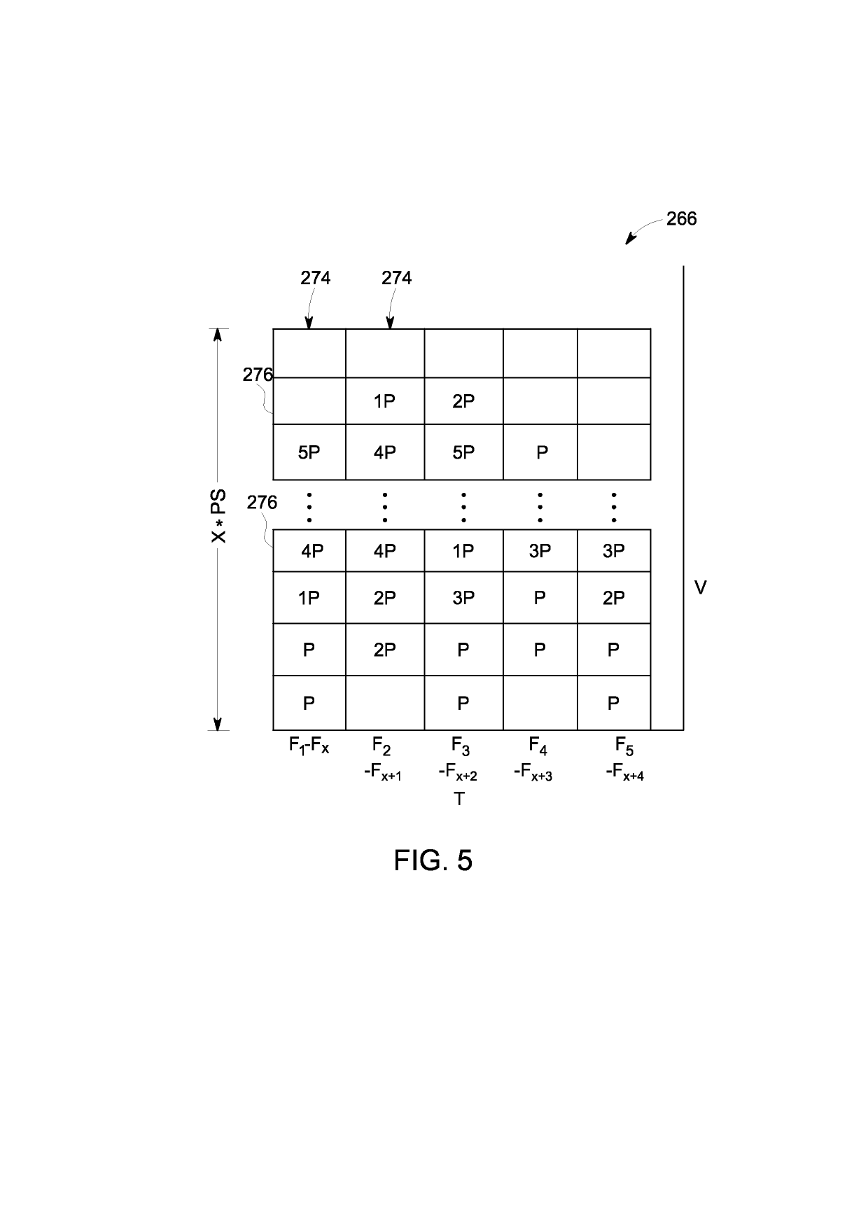

As indicated by step 206, module 40 generates control signals directing processor 30 to continue to transmit and receive sets or packets of size PS at each of the remaining locations L₂-Lₙ of the matrix of locations that are to form the region of interest for the color flow image. These transmit and receive events are not necessarily serial in nature but can be interleaved with each other. As indicated by step 208, module 40 directs processor 30 to process and analyze such signals to form the remaining pixels PL₂-PLₙ of the first frame of the color flow image. These sets of signals transmitted and received from the other locations of the region of interest are depicted in FIG. 5 as ultrasound signal packets or sets 304 and 306. Such pixels are presented on display 28 to form a first frame of the color flow image 60.

As indicated by step 210, color flow mode module 40 directs or instructs processor 30 to transmit and receive a first set of ultrasound signals of a packet or set size (PS) at the first location L₁ during a second frame F₂ of the color flow image. FIG. 5 illustrates the set or packet 308 of ultrasound signals direct at a location L1 during the second frame. As indicated by step 212, color flow mode module 40 (shown in FIG. 1) directs processor 30 to analyze such signals to produce an individual pixel PL₁ at location L₁ for the second frame. This pixel is part of a matrix of pixels that forms the second frame of the color flow image.

As indicated by step 214 and 216, this process or is repeated for each and every frame of the color flow image 60. For each frame from frame 1 to frame x, module 40 directs processor 30 to generate control signals causing transducer 24 to transmit and receive a packet or set of signals of size PS at each location L in the matrix of locations L that form the region of interest or area for color flow image 60. As a result, color flow mode module 40 generates a color flow image 60 formed from frames that are periodically refreshed using newly received ultrasound signals at each location which are represented by a corresponding refreshed pixel in the image 60. In one implementation, module 40 further stores the base data (there are semi-raw data used to form the frames of the color flow image 60) in data storage portion 46. As will be described hereafter, this data may be subsequently retrieved for subsequent generation of new spectrograms at any of various selected locations L from the stored color flow image 60.

Although steps 202-216 have been described as completing acquisition of all color data or all ultrasound signals for a single location of a frame prior to acquisition of color data or ultrasound signals for the next location in the frame or the next location in a successive frame, in other implementations, the acquisition of such ultrasound signals for a successive location may be initiated or started prior to the completion of acquisition of color data or ultrasound signals for the previous location. In particular, the acquisition of ultrasound signals or color data for a first location L may be temporally interleaved with the acquisition of ultrasound signals or color data for the one or more successive locations L.

For example, the pulse repetition frequency (the time between consecutive transmit-receive signals at a single location) may dictate a predetermined time delay between consecutive transmit-receive acquisitions at a first location. During this time delay, transmit-receive acquisitions may be completed at one or more other pixel or color image locations before the next successive transmit-receive acquisition of a packet of transmit-receive acquisitions is made at the first location. By way of a more specific example, transducer 24 may emit and receive a first pulse or signal and then (after a potential delay time) proceed to emit and receive a first pulse or signal at a second location, then proceed to emit and receive a first pulse signal at a third location, and so on, prior to returning to the first location to emit and receive a second pulse or signal at the first location. This pattern is repeated until all the pulse signals of the packet have been acquired. As a result, the time required to acquire signal sets from multiple locations is reduced since the acquisition of such sets is concurrent or overlapping depending upon the pulse repetition frequency and the interleave group size (the number of other locations for which transmit and receive actions are completed prior to returning to the original location).

As indicated by step 218, shared signal mode module 44 generates a spectrogram, such a spectrogram 266 shown in FIG. 1. In one implementation, as with method 100, the spectrogram 266 formed by method 200 may be concurrently or simultaneously presented on display 28 with the color flow image 60 even while a frame of the color flow image 60 is being generated or refreshed. In other words, spectrogram 266 may be generated without having to freeze or slow down a frame rate of the color flow image 60 being generated under the direction of module 40. However, unlike method 100, method 200 utilizes sets or packets of signals directed at a single selected location L1 from parts or the whole of multiple color flow image frames (F₁-Fₓ) to form each individual spectral distribution bar for the location L1 as shown by FIG. 5.

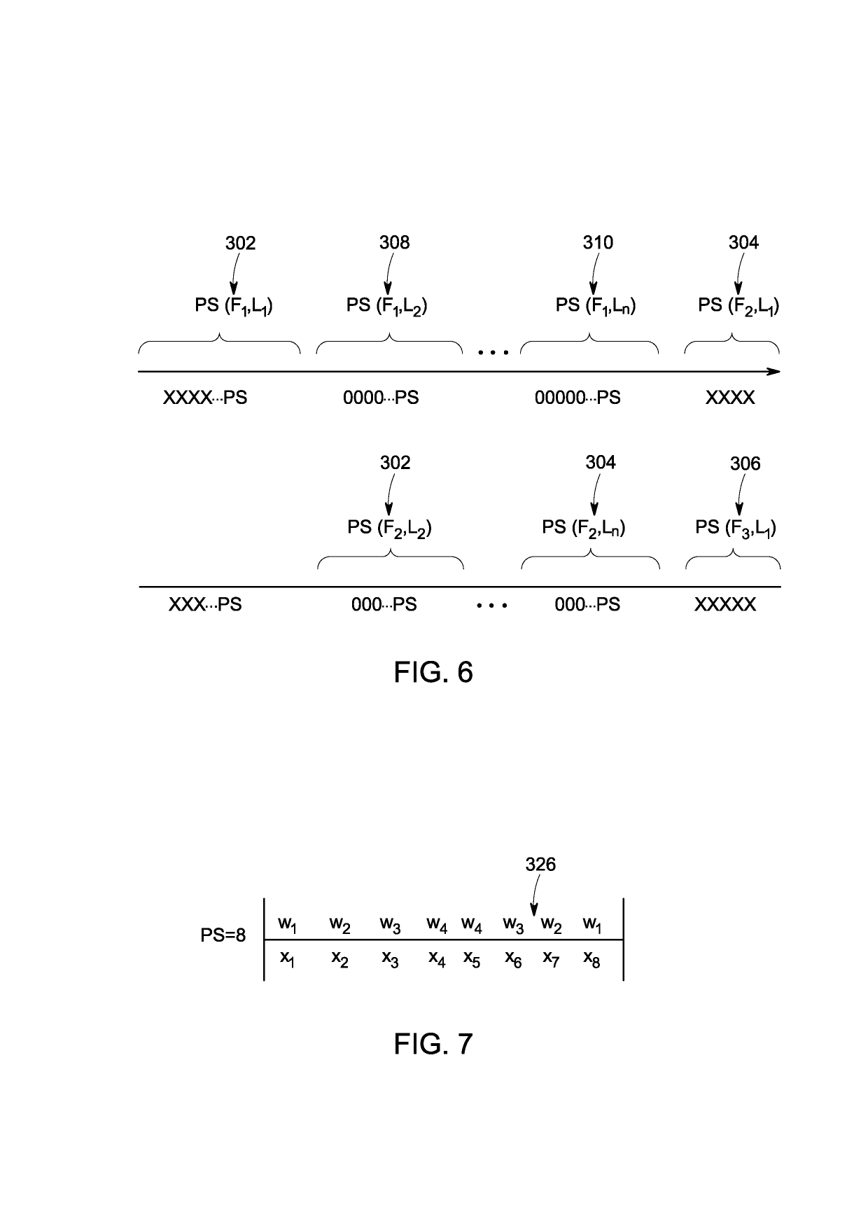

FIG. 6 is a diagram illustrating the processing of signals by processor 30 under the direction of module 44 to generate the (partial) spectrogram 266 shown in FIG. 5 during method 200. FIG. 6 illustrates the sets or packets 302, 304, 306 of ultrasound signals direct at a location L1 during the first frame, the second frame and the third frame, wherein the individual signals are pulses are represented by an “x”. FIG. 6 further illustrates the sequentially transmitted and received packets or sets of signals 308, 310, 312 and 314 from the other locations L₂ to Lₙ in the region of interest during each of frames F₁ to F₂. This pattern continues during the scanning of the different locations L of the region of interest of the color flow image for each frame of the color flow image.

As shown by FIG. 6, shared signal module 44 utilizes the transmitted and received sets or packets of signals for all locations L in the region of interest. As shown with sets 304 and 306 of signals, module 44 assigns a zero value to each of such signals that are representing reflections from locations other than the particular location L1 for which the spectrogram is being generated. These zero value signals serve as spacers, linking in time the signals x reflected from the particular location L₁ during the multiple frames F₁-Fₓ. As a result, transformation of the signals from location L₁ from the multiple frames out of the time domain to the frequency domain using such transforms as a fast Fourier transform or other transforms is facilitated.

Although FIG. 6 illustrates a number of zeros as being equal to the packet or signal set size PS for each location other than the particular location L1 for which the spectrogram is being generated, the number of zeros may vary depending upon the pulse repetition frequency and the interleave group size as discussed above. In particular, such zeros temporally space consecutive sets or packets of signals from different frames for the particular location for which the spectrogram is being generated. This spacing (and the corresponding number of zero signals) represents the time that has lapsed between acquisition of a first set of pack of signals for the first location for a first frame and the initiation of acquisition of the second set or pack of signals for the same first location for a second consecutive frame. This spacing may vary depending upon the number of locations to be sampled and the extent to which sets of signals for the other locations of a frame are acquired contemporaneously or in an overlapping or interleaved fashion with respect to the acquisition of all the signals for the packet or set of signals for the particular location (as well as the other locations). This extent may vary depending upon the selected pulse repetition frequency and interleave group size.

FIG. 7 the diagram illustrating one example method by which module 44 may process signals from the particular location for which the spectrogram 266 is to be generated. FIG. 7 illustrates an example of a single packet or set 320 of ultrasound signals directed at the same particular location L₁ during a frame. In the example illustrated, the set of signals has a packet size of eight. In other words, eight pulses or signals are transmitted or received from a particular location L₁ to form a pixel representing the location in the color flow image 60. The set 320 of pulses comprises a series of pulses or signals having an order based upon time. As shown by FIG. 7, each signal x is assigned a weight based upon its location in the time-based series of signals x₁-x₈. In the particular implementation, those particular signals closest to the ends of the series are assigned the least weight while those signals x closest to the middle are assigned the greatest weight. In the example shown, the outermost signals of the series x₁ and x₈ are assigned the lowest weight w₁, signals x₂ and x₇ are assigned a weight w₂ larger than weight w₁, signals x₃ and x₅ are assigned a weight w₃ larger than weight w₂ and signals x₄ and x₅ are assigned the greatest weight w₄. As a result, signal processing aberrations (frequency drop-offs) caused by the proximity of the outermost signals to the zeroed out signals for the other locations are avoided or minimized. In other implementations, other weighting schemes may be employed. In some implementations, such weighting schemes may be omitted.

As shown by FIG. 5 illustrating spectrogram 266, each spectral distribution bar 274 has a number of segments 276 based upon the number of pulses or signals in each packet or set of signals (PS), the time between frames (t), and the number of frames X from which signals for the particular location used to form the particular spectral distribution bar returned 74. For example, in one implementation, module 40 (shown in FIG. 1) may generate a color flow image 60 by transmitting and receiving 32 pulses or signals (PS=32) from each individual location of the matrix of locations forming the region of interest or the area of color flow image 60. Module 44 may generate spectral distribution bar 274 utilizing three frames (x=3). The frames may be separated to the time equal to firing 2 frames or 64 packets. In such an implementation, each vector distribution bar 274 would have 288 (3*(32+64)) segments or bins 276 using 96 ultrasound signals (the total number of signals from the location L1 during the three frames) and 192 zeros as input to the calculations for the 288 bins 276. As a result, the range of each bin 276 is reduced, enhancing frequency resolution of each bar 274 and a spectrogram 266. The larger the number of frames x utilized to form each spectral distribution bar 274, the greater the frequency resolution.

As shown by FIG. 5, in one implementation, the frames utilized to form the spectral distribution bars 274 are utilized in a sliding window fashion. For example, the first illustrated spectral distribution bar 274 is formed or using ultrasound signals from the first location during frames 1−x. The second illustrated spectral distribution bar 274 is formed or generated using frames 2−x+1 and so on. As a result, each distribution bar maybe formed from data based upon ultrasound signals over multiple frames without having to acquire signals for a completely new grouping of frames for each and every spectral distribution bar 274.

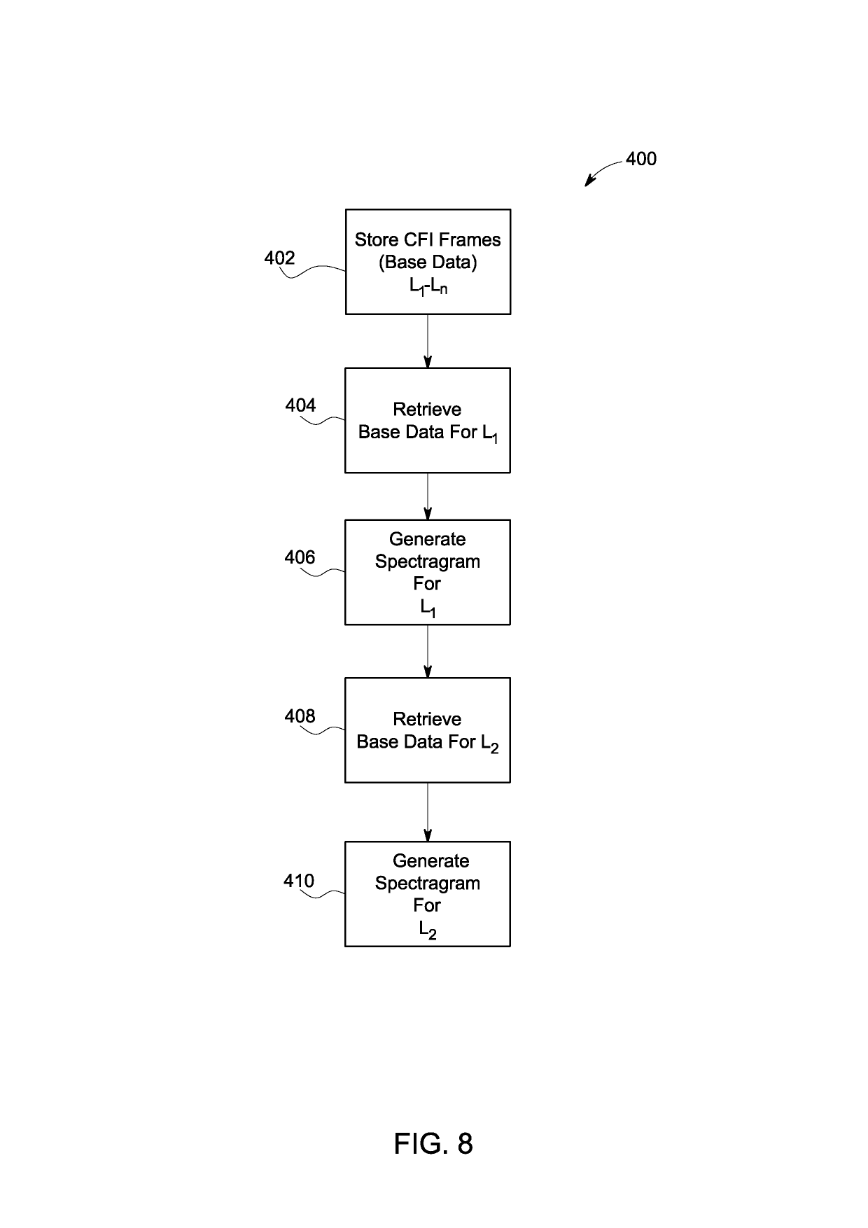

FIG. 8 is a flow diagram of an example method 400 that may be utilized by system 20. Method 400 enables a physician, caretaker or other person days, weeks, months or even years after the initial acquisition of ultrasound data to generate and view a spectrogram. Method 400 further enables such a person to generate a spectrogram at any of multiple locations from a single color flow image or cine loop of multiple color flow frames. As indicated by step 402, module 44 (shown in FIG. 1) stores the color flow image frames (their base data) for each of locations L₁-Lₙ of the region of interest or area of the color flow image 60. The color flow image frames and their base data may be stored in data region 46 of memory 32 (shown in FIG. 1). As indicated by step 404, in response to commands or instructions entered by input 26, processor 30, under the direction of module 44 may retrieve base data for a selected location L₁. As indicated by step 406, using such base data for the frames of the color flow image including location L₁, module 44 generates a spectrogram for the location L₁ pursuant to either step 106 in FIG. 2 or step 218 in FIG. 4 (described above). As indicated by steps 408 and 410, module 44 may generate additional spectrograms for other locations (such as location L₂) in the original region of interest or depicted area for color flow image 60 by retrieving base data from the stored color flow image frames. As a result, system 20 allows both live scanning and post processing evaluation.

Although the present disclosure has been described with reference to example embodiments, workers skilled in the art will recognize that changes may be made in form and detail without departing from the spirit and scope of the claimed subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example embodiments and set forth in the following claims is manifestly intended to be as broad as possible. For example, unless specifically otherwise noted, the claims reciting a single particular element also encompass a plurality of such particular elements.

What is claimed is:

1. A method comprising:
transmitting and receiving an ultrasound signal; and
sharing and using the same ultrasound signal to generate a color flow image and a spectrogram.

2. The method of claim 1 further comprising concurrently displaying the color flow image and the spectrogram.

3. The method of claim 1, wherein the ultrasound signal comprises a set of ultrasound pulses for a pixel for each frame of the color flow image.

4. The method of claim 3, wherein the set of ultrasound pulses is less than or equal to 32 ultrasound pulses.

5. The method of claim 3, wherein the spectrogram comprises a spectral distribution bar formed from and based upon the set of ultrasound pulses.

6. The method of claim 5, wherein the spectral distribution bar comprises less than or equal to 32 frequency or velocity bins.

7. The method of claim 5, wherein the set of ultrasound pulses comprises less than or equal to 32 ultrasound pulses.

8. The method of claim 1, wherein the color flow image comprises a plurality of frames, each frame comprising an array of pixels, each pixel based upon a set of pulses at a location of the image and wherein the spectrogram comprises a spectral distribution bar formed from and based upon a set of pulses, in part or in whole, from each of a plurality of frames for a pixel of the color flow image.

9. The method of claim 8 further comprising differently waiting values of the pulses in the set of pulses when generating the spectral distribution bar.

10. The method of claim 9, wherein the set of pulses comprises a series of the pulses having an order based upon a time that each pulse was received and wherein and pulses of the series are weighted less than intermediate pulses of the series.

11. The method of claim 8, wherein the set of pulses comprises less than or equal to 32 pulses.

12. The method of claim 1, wherein the color flow image has a frame rate of at least 5 Hz.

13. The method of claim 1 further comprising:
retrieving stored base data for a plurality of frames of a stored color flow image; and
generating a spectrogram from the retrieved base data.

14. The method of claim 1 further comprising generating the spectrogram from ultrasound signals corresponding to a plurality of temporally spaced apart frames of the color flow image.

15. An apparatus comprising:
an ultrasound transducer;
a controller configured to receive an ultrasound echo signal from the ultrasound transducer and to generate each of a color flow image and a spectrogram from the same ultrasound echo signal.

16. The apparatus of claim 15, wherein the controller is configured to simultaneously generate the color flow image and the spectrogram using the same ultrasound echo signal.

17. The apparatus of claim 15, wherein the controller is configured to generate a spectrogram after completion of ultrasound signal acquisition using stored base data, in part or in whole, for a plurality of frames of a stored color flow image.

18. The apparatus of claim 15, wherein the color flow image comprises a plurality of frames, each frame comprising an matrix of pixels, each pixel based upon a set of pulses at a location of the image and wherein the spectrogram comprises a spectral distribution bar formed from and based upon the base data, in part or in whole, for a set of pulses from each of a plurality of frames for a pixel of the color flow image.

19. An apparatus comprising:
a non-transient computer-readable medium storing code to direct a processor to generate each of a color flow image and a spectrogram from a same set of data derived from an ultrasound signal.

20. The apparatus of claim 19, wherein the color flow image comprises a plurality of frames, each frame comprising an matrix of pixels, each pixel based upon a set of pulses at a location of the image and wherein the spectrogram comprises a spectral distribution bar formed from and based upon the base data for a set of pulses from each of a plurality of frames for a pixel of the color flow image.
